(19) United States
(12) Patent Application Publication
(10) Pub. No.: US 2026/0256830 A1
(43) Pub. Date: Sep. 3, 2026

(54) PERITONEAL DIALYSIS FLUID

(71) Applicant: ZYTOPROTEC FLEXIBLE KAPITALGESELLSCHAFT, Wien (AT)

(72) Inventors: Alexandra JAINDL, Wien (AT); Christoph AUFRICHT, Klosterneuburg (AT); Klaus KRATOCHWILL, Wien (AT)

(21) Appl. No.: 18/878,563

(22) PCT Filed: Jul. 4, 2023
(86) PCT No.: PCT/EP2023/068317

§ 371 Date: Dec. 23, 2024

(30) Foreign Application Priority Data
Jul. 8, 2022 (EP) 22183830.3

(51) Int. Cl. A61K 33/06; A61K 9/08; A61K 33/00; A61K 47/02; A61K 47/12; A61K 47/18; A61K 47/26

(52) U.S. Cl. CPC A61K 33/06; A61K 9/08; A61K 33/00; A61K 47/02; A61K 47/12; A61K 47/183; A61K 47/26

(57) ABSTRACT

The invention concerns a peritoneal dialysis fluid comprising an amount of a first solution and an amount of a second solution, said amount of said first solution and said amount of said second solution having a predetermined ratio, said amount of said first solution and said amount of said second solution being mixed together upon administration, said fluid upon mixing of said amount of said first solution and said amount of said second solution having a pH of from 6.9 to 7.5.

The first solution is an acidic solution having a pH of 2.2 to 3.5 and contains an osmotic agent selected from the group consisting of glucose, glucose polymers or mixtures thereof.

The second solution is an alkaline buffer solution having a pH of 7.4 to 8.2 and contains a protective agent selected from the group consisting of L-glutamine, L-alanyl-L-glutamine, L-glutaminyl-L-alanine, L-glutaminyl-L-glycine, L-glycinyl-L-glutamine or mixtures thereof, as well as a lactate buffer in an amount of 30 to 45 mmol/L, calculated as amount in the fluid upon mixing said first and second solution.

The present invention relates to a peritoneal dialysis fluid (in the following also referred to as “PDF”).

Peritoneal dialysis fluids remove solutes and water from the uremic patient. The mechanism of PDFs is known and does not need to be elaborated in any more detail here.

PDF solutions comprise an osmotic agent. For the purposes of the present invention, the osmotic agent is glucose. PDFs containing glucose as the osmotic agent will in the following be referred to as “glucose-based” PDFs. The amount of glucose in such glucose-based PDFs is typically from about 0.5 to 4 g/L.

However, Glucose is known for being prone to degradation and forming glucose degradation products (GDP) ((1), EP 2 962 683 A1).

Therefore, it is known to store the glucose containing osmotic solution at rather acidic pH values (1).

Known embodiments of PDFs such as the “Dianeal® PDG4” product range manufactured by Messrs. Baxter (http://www.baxterpi.com/pi-pdf/Dianeal.PI.pdf, retrieved 23.06.2022) comprise one single glucose solution at a pH-value of around pH 5.0-5.5. However the extent of GDPs in such products is high.

Other embodiments therefore, employ an even lower pH-value of the osmotic solution, such as 3 to 5. However, peritoneal administration of a solution having a low pH value like this would not be possible or at least painful.

Thus, these further embodiments contain a second solution, which is an alkaline buffered solution, which is mixed with the osmotic solution before administration so as to form a final solution which is ready-to-use (“RTU”) having about a neutral pH value such as from 6.9 to 7.5.

The two solutions are manufactured at a predetermined ratio of their respective amount. They are stored separately but are mixed together upon administration. As a convenient means for storage, multi-chamber bags, especially double-chamber bags, containing the solutions in separated compartments which, however, can be connected with each other for mixing upon administration (e.g. by peelable seals), are known.

As a buffer, both lactate buffers and bicarbonate buffers and mixtures of the aforementioned are employed.

Various commercial solutions of the state of the prior art provide different approaches with regard to
the pH-values of the two solutions
the respective amounts of the two solutions as well as
the amount of buffer in the alkaline solution:

In the product range “Physioneal® 40 Glucose Clear-Flex” as manufactured by Messrs. Baxter, the glucose containing solution and the alkaline buffered solution are mixed in a ratio of 3:1. The alkaline buffered solution contains lactate buffer in an amount such that upon mixing of the solutions a total amount of 15 mmol/L results, and, furthermore, a bicarbonate buffer in an amount such that upon mixing of the solutions a total amount of 25 mmol/L results (https://www.baxter.ca/sites/g/files/ebysai1432/files/2018-12/Physioneal Clearflex EN.pdf, retrieved 24 Jun. 2022). The pH-values of the solutions are not disclosed but are assumed to be 2.1 (glucose containing solution) and >9 (alkaline buffer), respectively.

In the product range “Physioneal® 40 Glucose Viaflex” as also manufactured by Messrs. Baxter, the glucose containing solution and the alkaline buffered solution are mixed in a ratio of 725:1275. The alkaline buffered solution contains lactate buffer in an amount such that upon mixing of the solutions a total amount of 15 mmol/L results and, furthermore, a bicarbonate buffer in an amount such that upon mixing of the solutions a total amount of 25 mmol/L results. (https://www.baxter.ca/sites/g/files/ebysai1431/files/2018-11/Physioneal Viaflex EN.pdf, retrieved 24 Jun. 2022). The pH-values of the solutions are not disclosed but are assumed to be 4.2 (glucose containing solution) and 7.6 (alkaline buffer), respectively.

The product range “Balance®” as currently manufactured by Messrs. Fresenius also contains two chambers. The glucose containing solution and the alkaline buffered solution are mixed in a ratio of 1:1. The alkaline buffered solution contains lactate buffer in an amount such that upon mixing of the solutions a total amount of 35 mmol/L results.

The pH-values of the solutions are not disclosed but were measured on the basis of a sample product to be about 3.0 (glucose containing solution) and about 8.6 (alkaline buffer), respectively.

According to the product information relating to this product, sodium hydrogen carbonate is mentioned as an excipient.

In a brochure “Safety and biocompatibility in perfect balance” published by Messrs.

Fresenius in 2006 (2) a pH-value of the acidic solution of appr. 3.1 and a pH-value of the buffered solution of appr. 8.0 are reported. The mixing ratio of the two solutions is not disclosed.

Thus, from these prior art solutions it is known that in case of using a glucose solution with a low pH value such as 3 or even only 2 (which is favourable in terms of GDP formation), the pH value of the alkaline solution needs to be high (even 9 or more).

EP 2 962 683 A1, for the purposes of reducing GDP formation, proposes a fluid to be prepared by mixing of two or more solutions, wherein the sodium content is low and wherein no sodium ions are contained in the glucose containing solution. No pH-values are disclosed.

EP 1 465 688 B2, in the context of hemodialysis, employs a bicarbonate-based solution and an electrolyte (glucose) solution, and discloses several pH ranges for both solutions under moderate or extreme pH conditions.

Further state of the art discussing double chamber products for peritoneal dialysis is disclosed in EP 1 038 552, EP 1 131 077, EP 1 744 768, EP 2 647 397 and WO 2018/201443.

Several clinical and experimental observations have shown that PDF is cytotoxic, associated with a risk of technical failure of up to 30% with long term peritoneal dialysis (PD) treatment (2).

WO 2008/106702A1 provides a glucose-based peritoneal fluid containing a protective agent in the form of L-glutamine and dipeptides which are capable of releasing L-glutamine, said dipeptide being L-glutaminyl-L-glycine, L-glycinyl-L-glutamine, L-glutaminyl-L-alanine or L-alanyl-L-glutamine, or a mixture of two or more of said dipeptides, wherein the concentration of said dipeptide in the dialysis fluid is 2 mM to 25 mM.

According to this patent application, it was found that these dipeptides, especially L-alanyl-L-glutamine (in the following abbreviated as “Ala/Gln”), contribute to the prevention of Technical Failure.

Several publications have further investigated the effect of Ala/Gln in a glucose-based PDF ((3) to (27)).

In the following, the term L-alanyl-L-glutamine or “Ala/Gln” stands both for Ala/Gln as such as well as for all other protective agents within the scope of the present invention.

The problem underlying the present invention is to provide a glucose-based PDF containing said protective agent, especially Ala/Gln, and at the same time providing optimal results in terms of GDP formation.

This problem is solved by the subject matter of claim 1. Preferred embodiments are disclosed in the dependent claims.

### DETAILED DESCRIPTION OF THE INVENTION

In the context of formulating a ready-to-use glucose-based peritoneal dialysis fluid containing said protective agent, problems are encountered.

On the one hand, it was found that mixing of Ala/Gln and the glucose in one single solution would lead to reactions between the dipeptide and the glucose. Furthermore, at low pH values Ala/Gln itselfs tends to decompose.

Thus, a single-solution embodiment would not be storable over a longer period of time, which however is necessary for a PDF.

On the other hand, dipeptides such as Ala/Gln are known for not being stable under alkaline conditions ((28), (29)) and to form degradation products. This is even more the case given the need that a PDF must undergo a heat sterilisation of the solution at about 120° C.

However, as can be seen from the solutions discussed above, if a low pH-value for the glucose-containing solution is envisaged for reasons of minimizing GDP formation, the necessary pH value of the alkaline buffered solution needs to be high.

Surprisingly, it was found, however, that a PDF containing the two solutions as defined in claim 1 solves the problem underlying the invention.

The first solution provided by the present invention is a solution containing the osmotic agent glucose in a strongly acidic environment.

The second solution provided by the present invention is an alkaline buffered solution containing said protective agent, however at only a weakly alkaline pH-value.

Surprisingly, it was found that even if a low pH-value such as 2.2 to 3.5 of the glucose containing solution is employed (thus minimizing GDP formation), only a very low alkalinity of the alkaline buffered solution containing the Ala/Gln, such as 7.4 to 8.2, was sufficient to establish a neutral fluid upon mixing the first and the second solution. At the same time, the degradation of Ala/Gln upon storage can also be minimized due to the low alkalinity of the solution.

It needs to be noted that Ala/Gln itself would not be expected to have any effect on pH.

In a preferred embodiment of the present invention, the pH-value of said first solution is from 2.2 to 2.9, more preferred from 2.2 to 2.8, most preferred from 2.5 to 2.8.

In a further preferred embodiment in that the pH-value of said second solution is from 7.4 to 7.9, more preferred from 7.4 to 7.8, most preferred from 7.5 to 7.8.

The amount of lactate in said second solution is from 30 to 45 mmol/L, preferably from 34 to 41 mmol/L, most preferred about 35 mmol/L. All these values refer to the amount in the resulting (“ready-to-use”-RTU) fluid upon mixing said first and second solution. For example, if the mixing ratio of the first solution and the second solution is 1:1, with a target amount of 34 to 41 mmol/L in the RTU fluid, the amount of lactate in the second solution is from 68 to 82, preferably about 70 mmol/L.

This would resemble an amount of lactate such as known from the currently marketed “Balance®” product range as currently marketed by Messrs. Fresenius. However, while in that product the glucose containing solution is at a pH-value of 3.0 (i.e. less acidic than at least in the preferred embodiments of the present invention), the pH-value of the alkaline solution is about 8.6 (i.e. more alkaline than in the present invention), the respective amounts of both solutions being the same.

The lactate is preferably provided in the form of sodium lactate.

In the present invention, the ratio of the amount of said first solution to the amount of said second solution may be from 3:1 to 1:3, and is preferably from 2:1 to 1:2, even more preferred 1.5:1 to 1:1.5.

In an especially preferred embodiment, said ratio is about 1:1.

The term “amount” with regard to the first and the second solutions refers to the respective volumes of the solutions.

It is again surprising that when mixing quite equal to even absolutely equal amounts of the two solutions with each other, a neutral and, thus, physiologically acceptable PDF ready-to-be-used (“RTU”) results.

A further embodiment of the present invention is characterized in that both said first solution and said second solution contain one or more sodium compounds in a physiologically acceptable amount (calculated, again, on the resulting amount in the fluid upon mixing of the two solutions). In other prior art proposals, the sodium compounds are only present in one of the two solutions.

The total amount of sodium ions in both solutions may range from 130 mmol to 140 mmol/L.

The first solution of the PDF of the present invention preferably further contains a physiologically active agent selected from the group consisting of physiologically acceptable compounds containing sodium ions, calcium ions, magnesium ions and mixtures thereof.

The need for physiologically active agents comprising the afore-mentioned ions in a PDF is known to the skilled person and thus does not need to be discussed in more detail.

Typical physiologically active agents that may be employed in the fluid can be selected from sodium chloride, calcium chloride and magnesium chloride.

For the purposes of adjusting the pH-value of the first and second solution to the desired value, physiologically acceptable acids (for example HCl) and acceptable bases (for example NaOH) can be employed, respectively.

The amount of the protective agent, especially the amount of Ala/Gln preferably is from 2 mM to 25 mM, more preferably 5 mM to 10 mM, most preferably about 8 mM, again calculated as amount in the fluid upon mixing said first and second solution.

Preferably the amount of said protective agent after at least one year of storage, preferably after two years of storage, is 95% or more of its original amount. It has been found in long-time studies that the amount of degradation of Ala/Gln in the second solution indeed is very low.

Furthermore, it has also been found that the amount of glucose degradation, i.e. formation of GDP in said first solution during storage is low and is basically in the same range as that of commercially available products.

In an especially preferred embodiment said protective agent comprises, or even more preferably consists of L-alanyl-L-glutamine.

The two solutions of the PDF of the present invention may be manufactured by mixing the ingredients mentioned above in water, respectively, and heat sterilizing the resulting solutions.

Surprisingly, it has been found that the addition of only very small amounts of a bicarbonate compound to the second solution has apparently a further effect of stabilizing the protective agents against degradation, both during heat sterilization and during storage.

Furthermore, without wishing to be bound to theory, the addition of this amount of bicarbonate compound is believed to assist in achieving the target neutral pH-value upon mixing the two solutions.

Thus, in a further aspect the present invention provides a process for manufacturing the second solution of the PDF of the present invention, comprising the step of mixing together the ingredients of the second solution and, in addition, a bicarbonate buffer in an amount of 1 mmol/L to 6 mmol/L, calculated as amount in the fluid upon mixing said first and second solution, and sterilizing the mixed solution.

The amount of said bicarbonate buffer preferably is from 2 mmol/L to 4 mmol/L, even more preferably about 3 mmol/L.

This amount, again, is too small as to expect such a big influence of the pH-value upon mixing the two solutions with each other. Furthermore, this amount is even so small that the bicarbonate may no longer be detected in the second solution or in the fluid after mixing after a longer period of storage, such as 1 or 2 years. This is because of the equilibrium between the bicarbonate anion and CO₂, the latter possibly being able to penetrate through the film containing the solution at least partly.

Yet, it can be shown that the target neutral pH-value upon mixing the first and the second solution can also be obtained after longer storage time.

The bicarbonate compound preferably is sodium hydrogen carbonate.

The first and second solutions of the present invention may conveniently be filled into a bag comprising several chambers separated from each other by e.g. peelable seals, especially into a double chamber bag as previously described.

After filling into the bag, the solutions are sterilized, optionally further packaged with an overpoach, and stored.

### EXAMPLES

### Example 1—pH-Shift of Alanyl-Glutamine-Dipeptide (AGD) Containing Solutions after Sterilization

The pH value of AGD containing solutions was adjusted in 0.5 pH steps. These samples were used for analyzing the pH behaviour of the solutions during sterilization and to find the most appropriate combination of AGD containing alkaline solutions to reach the required pH range in the ready-to-use-solution (RTU solution). The final target composition of the AGD containing alkaline solutions in the RTU solution after mixing and the actual composition of the alkaline solution before mixing (based on a mixing ratio of acidic and alkaline solution of 1:1) were as follows:

TABLE 1
Composition of alkaline solution in the RTU solution.

| | Concentration in |
|---|---|
| Ingredient | RTU solution [mmol/L] |
| Sodium (Na⁺) | 132.00 |
| Lactate | 35.00 |
| AGD | 8.00 |

For the preparation of 1 L solution the following quantities of raw materials were weighed by means of a calibrated balance:

TABLE 2
Quantities of raw materials for preparing the alkaline solution.

| Raw material | Weighing-in [g/L] |
|---|---|
| Sodium chloride | 11.338 |
| Sodium lactate (60%) | 13.074 |
| AGD | 3.475 |

To prepare the AGD containing alkaline solutions, approximately 250 mL of deionized water were placed in a beaker. All ingredients were added with stirring and the solution was transferred to a plactic canister. While stirring deionized water was added and the pH value was adjusted with 1 M NaOH solution until the required value was met. Deionized water was filled up to the final volume of 1 L and pH was controlled. The required volume of solution was transferred to the bag through the filling tube. Sterilisation (steam-air mixture method) was performed with a standard programme for 5000 ml bags with sterilizable overwrapping under the conditions as shown in Table 3:

TABLE 3
Sterilisation programme for 5000 ml bags.
Chamber temperature profile

| | | |
|---|---|---|
| Target temperature | 123° | C. |
| Heating time up to target temperature | 20 | min |
| Holding time at target chamber temperature | 47 | min |
| Cooling time | 38 | min |

| | |
|---|---|
| Overall sterilisation cycle time | 1 h 45 min |

Solutions at six different pH-values were prepared (pH 6.0 to 8.5 in 0.5 steps) and pH values were determined before and after sterilisation for the determination of the pH shift after sterilisation.

All pH investigations were performed according to the current valid monograph 2.2.3 “Potentiometric Determination of pH” of the European Pharmacopoeia using a qualified pH electrode, calibrated for the corresponding pH range (alkaline, neutral or acidic) with certified NIST traceable buffer solutions pH 1.0, 4.0, 7.0, 9.0, and 10.0.

TABLE 4
Results of the pH-value evaluation of alkaline
solutions with AGD before and after sterilisation.
pH-value solution
(0.5 pH steps)
Alkaline solution with AGD

| pH before sterilisation | pH after sterilisation |
|---|---|
| 6.00 | 5.18 |
| 6.50 | 5.29 |
| 7.00 | 5.45 |
| 7.50 | 6.59 |
| 8.00 | 7.71 |
| 8.50 | 8.25 |

The results in Table 4 show a significant influence of the sterilisation process on the pH value of the alkaline solution of about 1 to 1.5 units within an acidic range of pH 6 to 7.5. The difference was smaller (approx. 0.3 units) at higher pH values of 8.00 and 8.50. These results indicate a superior stabilization of the pH value of the solutions by AGD at alkaline pH-values.

### Example 2—Stabilization of AGD by Addition of Small Amounts of a Bicarbonate Compound (Sodium Hydrogen Carbonate)

In order to determine the influence of sodium hydrogen carbonate on the pH shift during sterilisation, solutions with a constant bicarbonate content and varying pH values were prepared. The final concentration in the RTU solution was as follows:

TABLE 5
Composition of alkaline solution comprising sodium
hydrogen carbonate in the RTU solution.

| | Concentration in |
|---|---|
| Ingredient | RTU solution [mmol/L] |
| Sodium (Na⁺) | 132.00 |
| Lactate | 35.00 |
| AGD | 8.00 |
| Sodium hydrogen carbonate | 3.00 |

For the preparation of 1 L alkaline solution (again based on a 1:1 mixing ratio) the following quantities of raw materials were weighed by means of a calibrated balance:

TABLE 6
Quantities of raw materials for preparing the alkaline
solution comprising sodium hydrogen carbonate.

| Raw material | Weighing-in [g/L] |
|---|---|
| Sodium chloride | 10.987 |
| Sodium lactate (60%) | 13.074 |
| AGD | 3.475 |
| Sodium hydrogen carbonate | 0.504 |

Approximately 500 mL of deionized water were placed in a beaker. All ingredients were added with stirring and the solution was transferred to a plactic canister. While stirring, deionized water was added and the pH value was adjusted with 1 M NaOH solution until the required value was met. Deionized water was filled up to the final volume of 1 L and pH was controlled. The required volume of solution was transferred to the bag through the filling tube. Sterilisation was performed with a standard programme for 3000 ml bags with sterilized overwrapping.

TABLE 7
Sterilisation programme for 3000 mL bags.
Chamber temperature profile

| | | |
|---|---|---|
| Target temperature | 122.5° | C. |
| Heating time up to target temperature | 12 | min |
| Holding time at target chamber temperature | 54 | min |
| Cooling time | 58 | min |

| | |
|---|---|
| Overall sterilisation cycle time | 2 h 04 min |

The pH values were determined before and after sterilisation for the determination of the pH shift after sterilisation.

TABLE 8
Results of the pH-value evaluation of alkaline solutions with
AGD and NaHCO₃ before and after sterilisation.
pH-value solution
(0.2 pH steps)
Alkaline solution with AGD and NaHCO₃

| pH before sterilization | pH after sterilization |
|---|---|
| 7.20 | 7.23 |
| 7.40 | 7.41 |
| 7.60 | 7.62 |
| 7.80 | 7.78 |
| 8.00 | 7.96 |
| 8.20 | 8.15 |
| 8.40 | 8.35 |
| 8.60 | 8.54 |
| 8.80 | 8.65 |
| 9.00 | 8.80 |

The results in Table 8 exhibit no significant influence of the sterilization process on the pH-value of alkaline solutions in the presence of 3 mmol/L NaHCO₃. In comparison to Example 1, the presence of small amounts of bicarbonate obviously has an influence on the pH value during the sterilisation process. It is assumed, that the hydrogen carbonate has kind of a buffer effect on the solution.

### Example 3—Mixing of Acidic Solution and Alkaline Solution

For the pH mixing experiments acidic glucose containing and basic AGD containing solutions were prepared. In the first step, the pH value of the solutions were adjusted in 0.5 pH steps. These samples were used for analyzing the pH behaviour of the solutions during sterilization and to analyze the result of the combination of acidic and basic solutions with regard to the pH range in the mixed solution. As the target pH-value for the mixed, i.e. ready-to-use solution (RTU solution), a range of 6.9 to 7.5 was set.

TABLE 9
Composition of acidic solution in the RTU solution.

| | Concentration in |
|---|---|
| Ingredient | RTU solution [mmol/L] |
| Calcium (Ca²⁺) | 1.25 |
| Magnesium (Mg²⁺) | 0.5 |
| Glucose(C₆ H₁₂ O₆) | 214.0 |

TABLE 10
Quantities of raw materials for preparing the acidic solution.

| Raw material | Weighing-in [g/L] |
|---|---|
| Calcium chloride dihydrate | 0.367 |
| Magnesium chloride hexahydrate | 0.204 |
| Glucose monohydrate | 84.817 |

The pH-value of the acidic solution was adjusted by adding 1 M HCl until the desired pH-value was reached.

The alkaline solutions were prepared as described in Example 2. The ready-to-use solutions were prepared by mixing of samples (acidic and basic) with a ratio of 1:1 followed by an immediate analysis of the pH value.

TABLE 11
Results of mixing the solutions, with the pH-value of the acidic solution
indicated in the first column of the table, and the pH-value of the
alkaline solution being indicated in the first row of the table.

| pH-value | 6.00 | 6.50 | 7.00 | 7.50 | 8.00 | 8.50 |
|---|---|---|---|---|---|---|
| 2.00 | 4.46 | 4.48 | 4.51 | 4.59 | 4.80 | 5.37 |
| 2.50 | 4.84 | 4.90 | 4.95 | 5.15 | 6.71 | 7.87 |
| 3.00 | 5.06 | 5.13 | 5.23 | 5.73 | 7.52 | 8.12 |
| 3.50 | 5.16 | 5.24 | 5.38 | 6.23 | 7.63 | 8.19 |

The results in Table 11 show that the target pH range of 6.9 to 7.5 could not be reached after mixing acidic solution and alkaline solution

### Example 4—Mixing of Acidic Solution and Alkaline Solution

In a further trial, the pH values were set as as follows:
Acidic solutions pH 2.0 to 3.4 (0.2 steps)
Alkaline solutions pH 7.6 to 9.0 (0.2 steps)

Additionally, 3 mmol/L NaHCO₃ (referred as to the ready-to-use-solution) was added to the alkaline solution before the adjustment of the pH value.

The acidic solutions were prepared as described in Example 3 in a pH range from 2.0 to 3.4 in 0.2 steps. Basic solutions were prepared according to Example 2, and pH-values were adjusted in a range of 7.2 to 9.0 in 0.2 steps. Sterilistion of both solutions was performed with the standard programme for 3000 mL bags as shown in Table 7.

The ready-to-use solutions were prepared by mixing acidic and alkaline solutions with a ratio of 1:1 followed by an immediate analysis of the pH value.

TABLE 12
pH-values after mixing acidic and alkaline solutions (+NaHCO₃).

| pH-value | 7.2 | 7.4 | 7.6 | 7.8 | 8.0 | 8.2 | 8.4 | 8.5 | 8.7 | 8.8 |
|---|---|---|---|---|---|---|---|---|---|---|
| 2.0 | 4.91 | 4.95 | 5.10 | 5.34 | 5.63 | 6.11 | 6.66 | 7.15 | 7.36 | 7.62 |
| 2.2 | 5.36 | 5.58 | 5.91 | 6.29 | 6.71 | 7.18 | 7.58 | 7.84 | 7.94 | 8.09 |
| 2.4 | 6.03 | 6.26 | 6.58 | 7.01 | 7.40 | 7.68 | 7.93 | 8.12 | 8.21 | 8.33 |
| 2.6 | 6.55 | 6.68 | 7.00 | 7.35 | 7.62 | 7.84 | 8.06 | 8.24 | 8.33 | 8.46 |
| 2.8 | 6.78 | 7.00 | 7.28 | 7.54 | 7.78 | 7.97 | 8.18 | 8.35 | 8.44 | 8.57 |
| 3.0 | 6.96 | 7.19 | 7.43 | 7.66 | 7.86 | 8.04 | 8.24 | 8.42 | 8.51 | 8.64 |
| 3.2 | 7.05 | 7.25 | 7.50 | 7.71 | 7.90 | 8.07 | 8.26 | 8.43 | 8.52 | 8.67 |
| 3.4 | 7.10 | 7.34 | 7.54 | 7.72 | 7.92 | 8.09 | 8.29 | 8.46 | 8.56 | 8.70 |

The pH values marked in grey correspond to the pH range of 6.9 to 7.5, which has been defined as target range for the final product. Based on the results of the mixing tests, there are several possibilities regarding the adjustment of the pH values of acidic and alkaline solutions in order to achieve the desired target range of the pH value of the RTU solution of 6.9-7.5.

### Example 5—Examples for Acidic and Alkaline Solutions and Mixed Ready-to-Use Solution

Three examples for acidic and alkaline solutions, according to the invention, with different contents of glucose, respectively, are presented in the following. For all examples, the mixing ratio of acidic and alkaline solution is set at 1:1 (volume based).

TABLE 13
Composition of acidic solution and alkaline solution for a ready-
to-use solution (RTU solution) comprising 3.86% glucose.

| | | Amount |
|---|---|---|
| | [mmol/L] | [g/L] |
| Composition acidic Solution, pH = 2.6 | | |
| Glucose monohydrate, C₆H₁₂0₆ × H₂0 | 428.00 | 84.817 |
| Sodium chloride, NaCI | 186.02 | 10.872 |
| Calcium chloride dihydrate, CaCl₂ × 2 H₂O | 2.50 | 0.368 |
| Magnesium chloride hexahydrate, MgCl₂ × 6 H₂0 | 1.00 | 0.203 |
| Hydrochloric acid, HCI - q.s. - approx. | 0.87 | 0.032 |
| Composition alkaline Solution, pH = 7.7 | | |
| N(2)-L-Alanyl-L-Glutamin, C₈H₁₅N₃O₄ | 16.00 | 3.476 |
| Sodium lactate solution (50%), C₃H₅NaO₃ | 70.00 | 15.688 |
| Sodium hydrogen carbonate, NaHCO₃ | 6.00 | 0.504 |
| Sodium hydroxide, NaOH - q.s. - approx. | 1.98 | 0.079 |

TABLE 14
Composition of acidic solution and alkaline solution for a ready-
to-use solution (RTU solution) comprising 2.27% glucose.

| | | Amount |
|---|---|---|
| | [mmol/L] | [g/L] |
| Composition acidic Solution, pH = 2.6 | | |
| Glucose monohydrate, C₆H₁₂0₆ × H₂0 | 252.00 | 49.939 |
| Sodium chloride, NaCI | 186.02 | 10.872 |
| Calcium chloride dihydrate, CaCl₂ × 2 H₂O | 2.50 | 0.368 |
| Magnesium chloride hexahydrate, MgCl₂ × 6 H₂0 | 1.00 | 0.203 |
| Hydrochloric acid, HCI - q.s. - approx. | 0.87 | 0.032 |
| Composition alkaline Solution, pH = 7.7 | | |
| N(2)-L-Alanyl-L-Glutamin, C₈H₁₅N₃O₄ | 16.00 | 3.476 |
| Sodium lactate solution (50%), C₃H₅NaO₃ | 70.00 | 15.688 |
| Sodium hydrogen carbonate, NaHCO₃ | 6.00 | 0.504 |
| Sodium hydroxide, NaOH - q.s. - approx. | 1.98 | 0.079 |

TABLE 15
Composition of acidic solution and alkaline solution for a ready-
to-use solution (RTU solution) comprising 1.36% glucose.

| | | Amount |
|---|---|---|
| | [mmol/L] | [g/L] |
| Composition acidic Solution, pH = 2.6 | | |
| Glucose monohydrate, C₆H₁₂0₆ x H₂0 | 151.00 | 29.924 |
| Sodium chloride, NaCI | 186.02 | 10.872 |
| Calcium chloride dihydrate, CaCl₂ × 2 H₂O | 2.50 | 0.368 |
| Magnesium chloride hexahydrate, MgCl₂ × 6 H₂0 | 1.00 | 0.203 |
| Hydrochloric acid, HCI - q.s. - approx. | 0.87 | 0.032 |
| Composition alkaline Solution, pH = 7.7 | | |
| N(2)-L-Alanyl-L-Glutamin, C₈H₁₅N₃O₄ | 16.00 | 3.476 |
| Sodium lactate solution (50%), C₃H₅NaO₃ | 70.00 | 15.688 |
| Sodium hydrogen carbonate, NaHCO₃ | 6.00 | 0.504 |
| Sodium hydroxide, NaOH - q.s. - approx. | 1.98 | 0.079 |

TABLE 16
Composition or RTU solution upon mixing of acidic
and alkaline solution in a ratio of 1:1

| Composition of the ready- | Concentration | | Concentration |
|---|---|---|---|
| to-use (RTU) solution | [mmol/L] | | [g/L] |
| Anhydrous glucose | Glucose | 214.00 | 38.554 |
| (C₆H₁₂O₆) | 3.86% | | |
| | Glucose | 126.00 | 22.700 |
| | 2.27% | | |
| | Glucose | 75.5 | 13.035 |
| | 1.36% | | |
| Sodium (Na⁺) | 132.00 | | 3.035 |
| Calcium (Ca²⁺) | 1.25 | | 0.050 |
| Magnesium (Mg²⁺) | 0.50 | | 0.012 |
| Chloride (Cl⁻) | 96.87 | | 3.434 |
| Bicarbonate (HCO₃⁻) | 3.00 | | 0.183 |
| Lactate (C₃H5O3⁻) | 35.00 | | 3.117 |
| Alanyl-glutamine-dipetide | 8.00 | | 1.738 |
| (C₈H₁₅N₃O₄) | | | |

The pH value of the mixed, i.e. RTU solution in each case was in the targeted pH-range of 6.9 to 7.5.

### Example 6—Long-Term Stability of AGD

The long-term stability tests of AGD in an alkaline solution according to the present invention have been performed. Two sample solutions with pH values of 7.4 and 7.8 respectively, were prepared containing the same amounts, respectively, of AGD (16 mmol/L), NaHCO₃ (6 mmol/L) and sodium lactate (70 mmol/L). The pH of each sample was adjusted with 1 M NaOH. Aliquots of each sample were prepared under controlled conditions and stored under the defined conditions at 25° C./60% rH.

Sterilization was performed under following conditions:

TABLE 17
Sterilisation programe for stability tests.
Chamber temperature profile

| | | |
|---|---|---|
| Target temperature | 122.5° | C. |
| Heating time up to target temperature | 14 | min |

| | |
|---|---|
| Holding time at target chamber temperature | 1 h 02 min |
| Cooling time | 1 h 01 min |
| Overall sterilisation cycle time | 2 h 17 min |

The stability of AGD has been investigated by AS analysis. Analysis was performed by qualitative amino acid analysis with JEX and post column derivatization with Ninhydrine.

Both investigated samples showed the same trend in terms of only a very slight decrease of the AGD (less than 5% of original amount) after storage at 25° C./60% rH during a storage time of 52 weeks.

The least decrease in AGD was found in the sample with a pH-value of 7.4 (98% of original amount), with the decrease in AGD in the sample with a pH-value of 7.8 being>95% of the original amount.

Non-patent literature cited in the description:
(1) How to avoid glucose degradation products in peritoneal dialysis fluids. Perit Dial Int. 2006 July; 26(4):490-7.
(2) Safety and biocompatibility in perfect balance. Fresenius Medical Care Deutschland GmbH. 2006.
(3) What really happens to people on long-term peritoneal dialysis?Kidney Int. 1998 Dec;54(6):2207-17.
(4) A randomized controlled trial of alanyl-glutamine supplementation in peritoneal dialysis fluid to assess impact on biomarkers of peritoneal health. Kidney Int. 2018 Dec;94(6):1227-1237.
(5) Biomarker research to improve clinical outcomes of peritoneal dialysis: consensus of the European Training and Research in Peritoneal Dialysis (EuTRiPD) network. Kidney Int. 2017.
(6) Functional and Transcriptomic Characterization of Peritoneal Immune-Modulation by Addition of Alanyl-Glutamine to Dialysis Fluid. Sci Rep. 2017 Jul. 24; 7(1):6229
(7) Addition of Alanyl-Glutamine to Dialysis Fluid Restores Peritoneal Cellular Stress Responses —A First-In-Man Trial. PloS One. 2016 Oct. 21; 11(10):e0165045.
(8) Dynamic O-linked N-acetylglucosamine modification of proteins affects stress responses and survival of mesothelial cells exposed to peritoneal dialysis fluids. J Am Soc Nephrol. 2014 December; 25(12):2778-88.
(9) Interleukin-1 receptor-mediated inflammation impairs the heat shock response of human mesothelial cells. Am J Pathol. 2011 April; 178(4):1544-55.
(10) Alanyl-glutamine dipeptide restores the cytoprotective stress proteome of mesothelial cells exposed to peritoneal dialysis fluids. Nephrol Dial Transplant. 2012 Mar;27(3):937-46.
(11) Peritoneal dialysis fluids induce the stress response in human mesothelial cells. Perit Dial Int. 2001 Jan-Feb;21(1):85-8.
(12) Peritoneal dialysate fluid composition determines heat shock protein expression patterns in human mesothelial cells. Kidney Int. 2001 November; 60(5):1930-7.
(13) Alanyl-Glutamine Restores Tight Junction Organization after Disruption by a Conventional Peritoneal Dialysis. Biomolecules. 2020 Aug. 13; 10(8).
(14) The Peritoneal Surface Proteome in a Model of Chronic Peritoneal Dialysis Reveals Mechanisms of Membrane Damage and Preservation. Front Physiol. 2019 May 14; 10:472.
(15) Composite Outcome Improves Feasibility of Clinical Trials in Peritoneal Dialysis. Perit Dial Int. 2019 May 23.
(16) Does alanyl-glutamine supplementation offer potential to improve peritoneal dialysate biocompatibility?Kidney Int. 2018 December; 94(6):1050-1052.
(17) Injury-induced inflammation and inadequate HSP expression in mesothelial cells upon repeat exposure to dual-chamber bag peritoneal dialysis fluids. Int J Artif Organs. 2015 October;38(10):530-6.
(18) Is there such a thing as biocompatible peritoneal dialysis fluid?Pediatr Nephrol. 2016 Oct 8.
(19) HSP-mediated cytoprotection of mesothelial cells in experimental acute peritoneal dialysis. Perit Dial Int. 2010 May-Jun;30(3):294-9.
(20) Peritoneal dialysis fluids can alter HSP expression in human peritoneal mesothelial cells. Nephrol Dial Transplant. 2011 March;26(3):1046-52.
(21) Effects of epithelial-to-mesenchymal transition on acute stress response in human peritoneal mesothelial cells. Nephrol Dial Transplant. 2008 November; 23(11):3494-500.
(22) Evidence for HSP-mediated cytoskeletal stabilization in mesothelial cells during acute experimental peritoneal dialysis. Am J Physiol Renal Physiol. 2007 Jan;292(1):F47-56.
(23) Overexpression of HSP-72 confers cytoprotection in experimental peritoneal dialysis. Kidney Int. 2004Dec;66(6):2300-7.
(24) The dipeptide alanyl-glutamine ameliorates peritoneal fibrosis and attenuates IL-17 dependent pathways during peritoneal dialysis. Kidney Int. 2016 Mar;89(3):625-35.
(25) Effects of Alanyl-Glutamine Treatment on the Peritoneal Dialysis Effluent Proteome Reveal Pathomechanism-Associated Molecular Signatures. Mol Cell Proteomics. 2018 March;17(3):516-532.
(26) Peritoneal Dialysis Fluid Supplementation with Alanyl-Glutamine Attenuates Conventional Dialysis Fluid-Mediated Endothelial Cell Injury by Restoring Perturbed Cytoprotective Responses. Biomolecules. 2020 Dec. 15; 10(12).
(27) Targeted Metabolomic Profiling of Peritoneal Dialysis Effluents Shows Anti-oxidative Capacity of Alanyl-Glutamine. Front Physiol. 2019 Jan. 21; 9:1961.
(28) Degradation kinetics of L-alanyl-L-glutamine and its derivatives in aqueous solution. Eur J Pharm Sci. 1999 January;7(2):107-12.
(29) Quantitative high-performance liquid chromatography-tandem mass spectrometry impurity profiling methods for the analysis of parenteral infusion solutions for amino acid supplementation containing L-alanyl-L-glutamine. J Chromatogr A. 2012 Oct. 12; 1259:111-20.

1. A peritoneal dialysis fluid comprising an amount of a first solution and an amount of a second solution, said amount of said first solution and said amount of said second solution having a predetermined ratio, said amount of said first solution and said amount of said second solution being mixed together upon administration, said fluid upon mixing of said amount of said first solution and said amount of said second solution having a pH of from 6.9 to 7.5,
said first solution being an acidic solution having a pH of 2.2 to 3.5 and containing an osmotic agent selected from the group consisting of glucose, glucose polymers or mixtures thereof, and
said second solution being an alkaline buffer solution having a pH of 7.4 to 8.2 and containing a protective agent selected from the group consisting of L-glutamine, L-alanyl-L-glutamine, L-glutaminyl-L-alanine, L-glutaminyl-L-glycine, L-glycinyl-L-glutamine or mixtures thereof, and
a lactate buffer in an amount of 30 to 45 mmol/L, calculated as total amount in the fluid upon mixing said amount of said first solution and said amount of said second solution.

2. The fluid according to claim 1, wherein the pH-value of said first solution is from 2.2 to 2.9.

3. The fluid according to claim 1, wherein the pH-value of said second solution is from 7.4 to 7.9.

4. The fluid according to claim 1, wherein said amount of said lactate buffer is from 34 to 41 mmol/L, or about 35 mmol/L.

5. The fluid according to claim 1, wherein the ratio of the amount of said first solution to the amount of said second solution is from 3:1 to 1:3.

6. The fluid according to claim 5, wherein said ratio is about 1:1.

7. The fluid according to claim 1, wherein both said first solution and said second solution contain one or more sodium compounds in a physiologically acceptable amount, calculated as total amount in the fluid upon mixing said amount of said first solution and said amount of said second solution.

8. The fluid according to claim 1, wherein said first solution further contains a physiologically active agent selected from the group consisting of physiologically acceptable compounds containing sodium ions, calcium ions, magnesium ions and mixtures thereof.

9. The fluid according to claim 1, wherein the amount of said protective agent is from 2 mM to 25 mM, calculated as amount in the fluid upon mixing said first and second solution.

10. The fluid according to claim 1, wherein the amount of said protective agent in said second solution after at least one year of storage is 95% or more of its original amount.

11. The fluid according to claim 1, wherein said protective agent is or comprises L-alanyl-L-glutamine.

12. A process for manufacturing the second solution of the fluid according to claim 1, comprising the step of mixing together the ingredients of the second solution and, in addition, a bicarbonate compound in an amount of 1 mmol/L to 6 mmol/L, calculated as amount in the fluid upon mixing said first and second solution, and sterilizing the mixed solution.

13. A process according to claim 12, wherein said amount of said bicarbonate compound is from 2 mmol/L to 4 mmol/L.

14. The fluid according to claim 2, wherein the pH-value of said first solution is from 2.2 to 2.8, or from 2.5 to 2.8.

15. The fluid according to claim 3, wherein the pH-value of said second solution is from 7.4 to 7.8, or from 7.5 to 7.8.

16. The fluid according to claim 5, wherein the ratio of the amount of said first solution to the amount of said second solution is from 2:1 to 1:2.

17. The fluid according to claim 5, wherein the ratio of the amount of said first solution to the amount of said second solution is from 1.5:1 to 1:1.5.

18. The fluid according to claim 9, wherein the amount of said protective agent is from 5 mM to 10 mM, or about 8 mM, calculated as amount in the fluid upon mixing said first and second solution.

19. The fluid according to claim 1, wherein the amount of said protective agent in said second solution after two years of storage is 95% or more of its original amount.

20. A process according to claim 13, wherein said amount of said bicarbonate compound is about 3 mmol/L.
